(19) United States
(12) Patent Application Publication
(10) Pub. No.: US 2004/0064164 A1
(43) Pub. Date: Apr. 1, 2004

(54) Connector module jumper for quadrapolar leads

(76) Inventors: Andrew J. Ries, Lino Lakes, MN (US); Timothy W. Holleman, Ham Lake, MN (US)

Correspondence Address: MEDTRONIC, INC., 710 MEDTRONIC PARKWAY NE, MS-LC340, MINNEAPOLIS, MN, 55432-5604, US

(21) Appl. No.: 10/256,726

(22) Filed: Sep. 30, 2002

(51) Int. Cl. A61N 1/375

(52) U.S. Cl. 607/37

(57) ABSTRACT

A dual chamber implantable cardioverter defibrillator (ICD) is configured for enabling quadrapolar dual lead systems, having differing lead connector configurations, to properly function in corresponding quadrapolar ports of the ICD. A header assembly, mounted on the ICD provides an electrical junction point between the cardiac leads and electrical circuitry in the ICD. The header assembly includes at least two ports therein, wherein each port includes a first and a second substantially longitudinal bore. Terminal blocks are located in the first bores, and spring clip contacts are located in the second bores. Electrical isolation is provided between electrical contacts located in the ports and corresponding lead connectors. A jumper connection includes electrical connection to certain spring clip contacts, one contact being located in a separate port. Subsequently, cardiac leads with differing lead connectors may be inserted into the ports to enable proper function.

### FIELD OF THE INVENTION

The present invention relates to a dual chamber, implantable cardioverter defibrillators (ICDs), and lead compatibility. More particularly, the invention relates to quadrapolar connector ports in these ICDs, and configuring the ports so that quadrapolar leads of differing conductor configurations will properly function therein.

### BACKGROUND OF THE INVENTION

Implantable pacemakers and cardioverter defibrillators (ICDs) are electronic medical devices that monitor the electrical activity of the heart and provide electrical stimulation to one or more heart chambers, when necessary. For example, a pacemaker senses an arrhythmia, i.e., a disturbance in heart rhythm, and provides appropriate electrical stimulation pulses, at a controlled rate, to selected chambers of the heart in order to correct the arrhythmia and restore the proper heart rhythm. The type of arrhythmias that may be detected and corrected by pacemakers include bradycardias, which are unusually slow heart rates, and certain tachycardias, which are unusually fast heart rates.

Implantable cardioverter defibrillators (ICDs) also detect arrhythmias and provide appropriate electrical stimulation pulses to selected chambers of the heart to correct the abnormal heart rate. In contrast to pacemakers, however, the pulses from an ICD can be much stronger and less frequent. This is because ICDs are generally designed to correct fibrillations, which are rapid, unsynchronized quiverings of one or more heart chambers, and severe tachycardias, where the heart beats are very fast but coordinated. To correct such arrhythmias, an ICD delivers a low, moderate or high energy shock to the heart. In addition to functioning as a cardioverter defibrillator, some ICDs are designed to provide pacing support to the heart. Such ICDs sense the occurrence of a cardiac arrhythmia and automatically apply an appropriate therapy to the heart aimed at terminating the specific arrhythmia detected.

The pacing functions of ICDs are described as either single-chamber or dual-chamber systems. A single-chamber ICD uses one outgoing lead, which may be placed in the right atrium or right ventricle. A dual chamber system uses two outgoing leads, typically with one lead being placed in the right atrium and the other lead being placed in the right ventricle. These leads generally sense or stimulate the different chambers of the heart that they lie within, and are connected to the ICD through a header assembly which contains ports for end connectors on the leads. Header assemblies, provided for electrically coupling the leads to the electronic circuitry contained within the ICD, are well known in the art. Representative examples of such header assemblies appear in U.S. Pat. No. 5,545,188, issued to Bradshaw et al. and U.S. Pat. No. 4,934,366, issued to Truex et al., both of which are incorporated herein by reference. Such header assemblies are generally constructed of two opposing half-bodies that are connected and sealed to form the header housing.

Standards have been created by the International Standards Organization (ISO) to conform lead connector and header port design. A lead having a connector conforming to such an international standard will generally have a designation to indicate that it was manufactured in compliance with the standard guidelines. Two examples of leads with this designation are IS-1 (ISO 5841.3:1992) and DF-1 (ISO 11318:1993). While each international standard is different in regards to its own particular guidelines, it has at least become understood that when leads conform to a particular standard, it enables them to be interchangeable with each other. For example, when using an ICD that has IS-1 connector ports contained within its header assembly, leads produced by any manufacturer may be used and should be interchangeable with each other, as long as they meet the respective IS-1 international standard. Likewise, if an ICD has DF-1 connector ports contained within its header assembly, leads produced by any manufacturer may be used and should be interchangeable with each other, again so long as they meet the respective DF-1 international standard.

Leads may be unipolar, in which case they are constructed with a single conductor wire, typically coiled around a central axis and encased in a sleeve of insulation. In addition, leads may have multiple conductors, where each conductor is separately insulated from each other, with all the leads typically still being encased in a single sleeve of insulation. Examples of such include bipolar, tripolar, and quadrapolar leads. Whether being single or multi-polar, leads have their corresponding conductors electrically connected to distinct electrodes at the distal end of the lead that either sense or stimulate a particular heart region. For example, a tripolar lead will generally be comprised of three electrodes, which may include two electrodes (e.g., a tip electrode and ring electrode) utilized for low voltage sensing or pacing, and one electrode (e.g., a coil electrode) utilized for high voltage defibrillation. In contrast, a bipolar lead will generally be comprised of only two electrodes (e.g., a tip electrode and a ring electrode), both utilized for low voltage sensing or pacing. IS-1 and DF-1 connectors generally have two and one conductor wires, respectively.

As previously discussed, leads are used to sense or stimulate the heart through the use of electrodes. By definition, leads are considered to be high voltage (HV) if they contain any high voltage electrodes, otherwise, they are considered to be low voltage (LV). HV and LV electrodes are each designed for a distinct function. While HV electrodes are designed to fight fibrillation and organized tachycardia episodes, LV electrodes are designed to pace or sense arrhythmias. A DF-1 connector is configured for high voltage, whereas a IS-1 connector is configured for low voltage. Thus, these two connector types, even though they may have some similarities in electrode layout, are not interchangeable. One would obviously not want to place high voltage on a conductor designed for low voltage. Such an event may cause an unsafe environment for the patient. Thus, there are safeguards in place that guard against this scenario from happening. For instance, if one attempts to insert an IS-1 connector (LV) into a DF-1 connector port on an ICD header, the IS-1 connector (LV), because of the size of its lead connector, would be physically locked out of the DF-1 connector port and thus, not insert to full depth, thereby, not allowing the IS-1 lead to function.

The problem the present invention addresses is in relation to leads typically used in modern dual chamber ICD system applications. Current applications use quantities of IS-1 and DF-1 connectors for every ICD. More modern ICDs can utilize a single connector system having only a single plug for multiple leads, each going to distinct chambers of the heart. For example, a typical connector system will have outgoing leads to both the ventricular and atrial chambers while only having a single plug comprised of generally two lead connectors going into two corresponding ports in the ICD header assembly. One advantage of providing such a system is that it is more convenient for a physician to deal with only one plug being connected to an ICD rather than a variety of separate lead connectors. However, these lead systems may prove to have disadvantages as well.

The quadrapolar standard is an example of one particular international standard being developed that involves such a single plug connector system and corresponding header port design. For dual chamber ICDs employing a standard, six-output conductor system using quadrapolar lead connectors, the lead systems may be classified into two categories. One category of lead systems for such DC ICDs is quadrapolar-bipolar (4-2). The other category of lead configuration is tripolar-tripolar (3-3). These configurations indicate the number of electrodes stemming from the four connectors of the ventricular-atrial leads, respectively. One problem that may occur in dealing with quadrapolar lead systems is the expectation by people familiar with the compatibility of the IS-1 and DF-1 leads that all quadrapolar lead systems are compatible as well. With the differing lead configurations (e.g., 4-2 and 3-3), this may not be the case. quadrapolar connectors are typically used only for lead configurations having more than two conductors (e.g., tripolar and quadrapolar leads). Under the 4-2 configuration, the bipolar lead would have an IS-1 connector. It is possible, however, for the bipolar lead to have an quadrapolar connector, where the third and fourth terminal contacts are “dead” contacts (i.e., unused contacts having no corresponding lead conductor or electrode). Under such a scenario, one could insert the quadrapolar connector from a 4-2 configuration into a header assembly designed for a 3-3 configuration. The fourth terminal contact on the ventricular lead would be left unused by the 3-3 header assembly. This uncertainty will likely cause public confusion about not only the new quadrapolar standard, but also may create confusion in working with the IS-1 and DF-1 leads thereafter. The present invention provides a way to avoid this confusion.

### BRIEF SUMMARY OF THE INVENTION

The present invention provides systems and methods for coupling differently configured quadrapolar cardiac lead systems to a cardiac stimulator by utilizing an inventive header assembly. One system involves a header assembly comprised of a housing. The housing has at least first and second ports extending from a side surface, where the ports are comprised of first and second substantially aligned longitudinal bores. The housing is adapted for sealably receiving a lead system plug comprised of at least two lead connectors. These lead connectors include a terminal pin and at least three terminal contacts. The header assembly is also comprised of an electrically conductive terminal block that is positioned within each first bore, where each terminal block is electrically connectable to the terminal pin of the respective lead connector and to the cardiac stimulator. The header assembly is also comprised of at least three electrically conductive spring clip contacts positioned within each second bore, where each spring clip contact is electrically connectable to a respective terminal contact and to the cardiac stimulator. The header assembly also is comprised of a electrically insulating seals positioned on both sides of each spring clip contact, including between the terminal block and the spring clip contact closest to such terminal block in each port. A jumper connection is used to electrically connect one of the spring clip contacts located in the first port to one of the spring clip contacts in the second port.

In another embodiment, a header assembly is configured to electrically connect cardiac leads to a cardiac stimulator. The header assembly includes a housing having elongate ports extending inward from a side surface of the housing. The ports are each adapted for removably receiving and sealing a lead connector where the lead connector has a terminal pin and at least three terminal contacts. The header assembly also includes an electrically conductive terminal block positioned within each port adapted for electrically connecting to the terminal pin of the lead and to the cardiac stimulator. The header assembly also has at least three electrically conductive spring clip contacts positioned within each port adjacent to the respective terminal block where each such spring clip contact electrically connects to a respective terminal contact and to the cardiac stimulator. The header assembly also is comprised of a plurality of seals positioned on both sides of each spring clip contact, including between the terminal block and its closest spring clip contact in each port. A jumper connection is used to electrically connect one of the spring clip contacts located in one of the ports to one of the spring clip contacts located in another one of the ports.

In yet another embodiment, a method is provided for constructing a header assembly for electrically connecting differently configured quadrapolar cardiac lead systems to a cardiac stimulator involves constructing a differently configured header assembly. A header assembly that is normally configured to accept an quadrapolar lead system plug is provided. The header assembly has at least two ports extending from its side surface, where the ports are comprised of first and second substantially aligned longitudinal bores. The ports are adapted for sealably receiving the lead system plug, where the lead system plug is comprised of at least two lead connectors. Each lead connector has a terminal pin and at least three terminal contacts. The housing of the header assembly is opened so that the second bores are visibly exposed. At least three electrically conductive spring clip contacts are contained within each second bore, where each spring clip contact is connectable to a respective terminal contact. A jumper connection is provided. One end of the jumper connection is electrically connected to a spring clip contact located in one port and the other end of the jumper connection is electrically connected to a spring clip located in the other port. The housing of the header assembly is then closed.

### BRIEF DESCRIPTION OF THE DRAWINGS

FIG. 1 is a pictorial view of an exemplary embodiment of a cardiac stimulator system in accordance with the present invention;

FIG. 2 is a pictorial view of another exemplary embodiment of a cardiac stimulator system in accordance with the present invention;

FIG. 3 is a cross-sectional view taken along the line 3-3 of FIG. 1 annotating several features of the header assembly of the invention;

FIG. 4 is a cross-sectional view taken along the line 3-3 of FIG. 1 annotating additional features of the header assembly of the invention;

FIG. 5 is a cross-sectional view taken along the line 3-3 of FIG. 1 annotating several features of an quadrapolar lead connector; and

FIG. 6 is a cross-sectional view taken along the line 6-6 of FIG. 2.

### DETAILED DESCRIPTION OF THE PREFERRED EMBODIMENTS

The following detailed description is to be read with reference to the figures, in which like elements in different figures have like reference numerals. The figures, which are not necessarily to scale, depict selected embodiments and are not intended to limit the scope of the invention. Skilled artisans will recognize that the examples provided herein have many useful alternatives that fall within the scope of the invention.

The present invention is not limited to dual chamber (DC) implantable cardioverter defibrillators (ICDs) and may be employed in many various types of electronic and mechanical devices for treating patient medical conditions. It is to be further understood, moreover, that the present invention may be employed in many other types of therapeutic or diagnostic ports and is not limited to ICD ports only. For purposes of illustration only, however, the present invention is below described in the context of DC ICDs.

In addition, the present invention is directed to solving a compatibility limitation with DC ICDs employing a standard, six-output conductor system using quadrapolar lead connectors. Of course, the present invention is not limited to merely this one configuration. The typical dual lead system for such six output devices may be broken into two distinct categories, 4-2 lead systems (quadrapolar-bipolar) and 3-3 (tripolar-tripolar) lead systems. These configurations indicate the number of electrodes stemming from the four connectors of the ventricular-atrial leads, respectively. Traditionally, the four signals on the ventricular lead connector of the 4-2 system are Vₜᵢₚ, V_{ring}, RV and SVC, where Vₜᵢₚ and V_{ring} are low voltage and RV and SVC are high voltage. The two signals on the atrial lead connector of the 4-2 system are Aₜᵢₚ and A_{ring}, where both signals are low voltage. In contrast, the three signals traditionally on the ventricular lead connector of the 3-3 system are Vₜᵢₚ, V_{ring} and RV. The SVC is, instead, traditionally located on the atrial lead connector along with Aₜᵢₚ and A_{ring}.

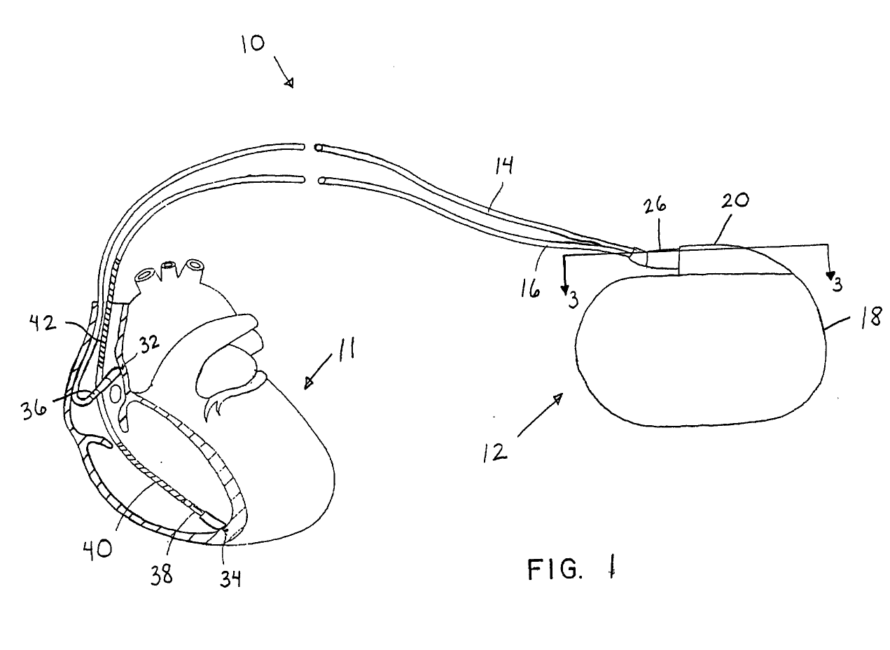

Turning now to the drawings, and in particular to FIG. 1, there is shown one embodiment of the present invention that includes an implantable cardiac stimulator system 10 that may be suitable for either endocardial or epicardial stimulation of a human heart 11. The cardiac stimulator system 10 includes an ICD 12 and a pair of cardiac leads, 14 and 16. Being of such a length that they are shown broken, lead 14 may generally be referred to as an atrial lead and lead 16 may generally be referred to as a ventricular lead. The ICD 12 consists of a can 18 composed of titanium, or like materials, connected to a header assembly 20. The can 18 encases the electronic components of the ICD 12, which may include storage cells, power transistors, microprocessors, telemetry circuits, sensors, and induction coils for rechargeable storage cells, among others. While the header assembly 20 is shown located outside the can 18, it is contemplated that the header assembly 20 may be located inside the can 18, and it is not meant to limit the invention in embodying the header assembly 20 as such in FIG. 1.

The proximal ends of the leads 14 and 16 are connected to the header assembly 20 through a dual lead system plug 26, comprised of two lead connectors. The distal ends of the leads 14 and 16 terminate in respective tip electrodes 32 and 34 that are designed to be attached to the tissue requiring electrical sensing or stimulation. The lead 14 is depicted in a bipolar configuration, and the ventricular lead 16 is depicted in a quadrapolar configuration. Other configurations are possible, however, and one such other configuration is discussed further below. Accordingly, in this depiction, the distal end of the lead 14 is provided with a tip electrode 32 and a ring electrode 36 used for low voltage sensing or pacing in the atrial chamber. The distal end of the lead 16 is provided with a tip electrode 34 and a ring electrode 38 used for low voltage sensing or pacing in the ventricular chamber, and a pair of coil electrodes 40 and 42 used for high voltage defibrillation in the ventricular chamber and the superior vena cava, respectively.

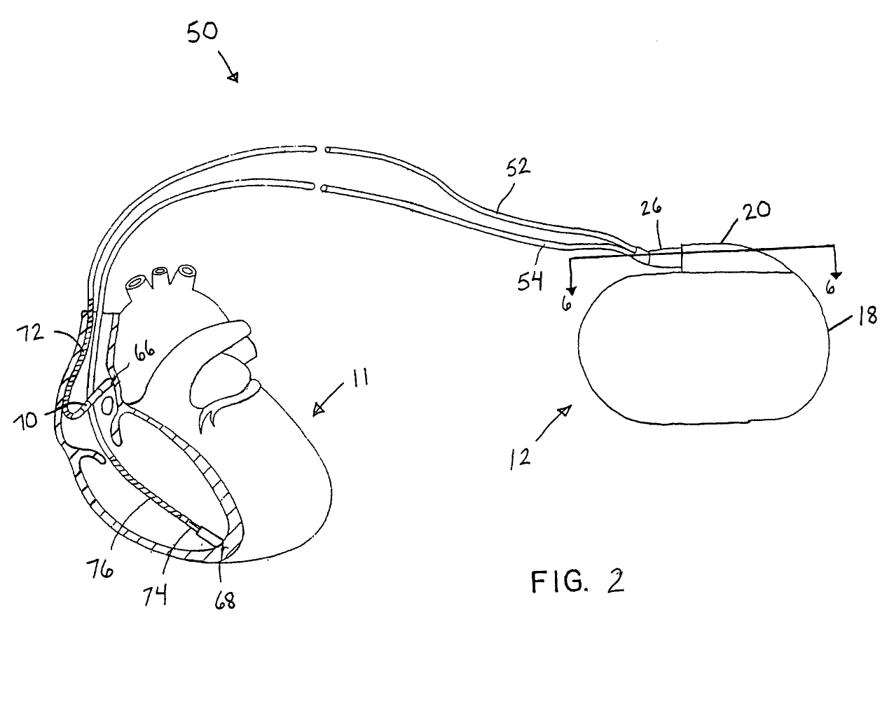

In comparison to FIG. 1, FIG. 2 shows a slightly different implantable cardiac stimulator system 50 that once again may be suitable for either endocardial or epicardial stimulation of a human heart 11. The cardiac stimulator system 50 includes the same ICD 12 mentioned in FIG. 1, but utilizes a different pair of cardiac leads, 52 and 54. Like in FIG. 1, the leads here, 52 and 54, being of such length that they are shown broken, may generally be referred to as an atrial lead and a ventricular lead respectively, and the ICD 12 consists of a can 18 composed of titanium, or like materials, connected to a header assembly 20. Also, while the header assembly 20 is again shown located outside the can 18, it is still contemplated that the header assembly 20 may be located inside the can 18, and it is not meant to limit the invention in embodying the header assembly 20 as such in FIG. 2.

The proximal ends of the leads 52 and 54 are connected to the header assembly 20 through a dual lead system plug 26, comprised of two lead connectors (not shown). The distal ends of the leads 52 and 54 terminate in respective tip electrodes 66 and 68 that are designed to be attached to the tissue requiring electrical sensing or stimulation. Both the leads 52 and 54 are depicted in a tripolar configuration. Accordingly, the distal end of the lead 52 is provided with the tip electrode 66 and a ring electrode 70 used for low voltage sensing or pacing in the atrial chamber, and a coil electrode 72 used for high voltage defibrillation in the superior vena cava. The distal end of the lead 54 is provided with the tip electrode 68 and a ring electrode 74 used for low voltage sensing or pacing in the ventricular chamber, and a coil electrode 76 used for high voltage defibrillation in the ventricular chamber as well.

As can be noted from the two embodiments illustrated, there is a 4-2 dual lead system configuration embodied in FIG. 1, and a 3-3 dual lead system configuration embodied in FIG. 2. The 4-2 lead configuration has a high voltage lead (4 electrodes) being paired with a low voltage lead (2 electrodes), whereas the 3-3 lead configuration has two high voltage leads (3 electrodes each) being paired together. One problem that may occur in dealing with quadrapolar lead systems is the expectation by people familiar with the compatibility of the IS-1 and DF-1 leads that all quadrapolar lead systems are compatible as well. With the differing lead configurations (e.g., 4-2 and 3-3), this may not be the case. quadrapolar connectors are typically used only for lead configurations having more than two conductors (e.g., tripolar and quadrapolar leads). Under the 4-2 configuration, the bipolar lead would have an IS-1 connector. It is possible, however, for the bipolar lead to have an quadrapolar connector, where the third and fourth terminal contacts are “dead” contacts (i.e., unused contacts having no corresponding lead conductor or electrode). Under such a scenario, one could insert the quadrapolar connector from a 4-2 configuration into a header assembly designed for a 3-3 configuration. The fourth terminal contact on the ventricular lead would be left unused by the 3-3 header assembly.

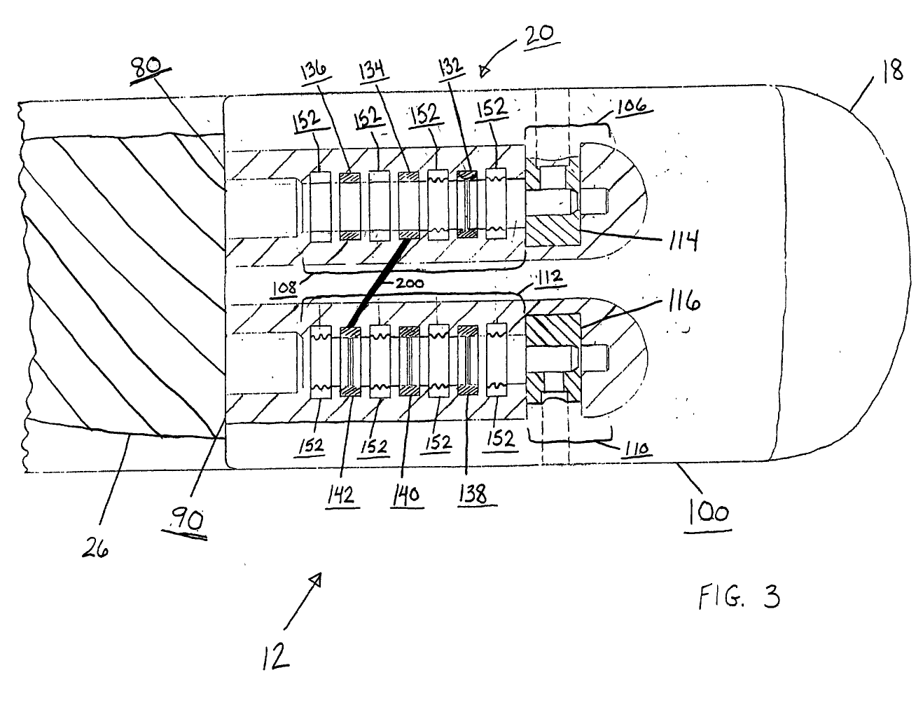

One motivation of the invention is to enable compatibility between quadrapolar dual lead system configurations with DC ICDs designed with quadrapolar ports. However, if compatibility is to be achieved, specifically in the aforementioned 4-2 versus 3-3 case, one must configure the quadrapolar ports to be able to “handle” either type of lead configuration. The detailed structure of the header assembly 20 and the connection thereof with proximal lead connectors of the respective leads 14 and 16 in FIG. 1 may be understood by referring now to FIGS. 3 through 5, which illustrate a cross-sectional view of FIG. 1 taken at line 3-3. Referring to FIG. 3, the header assembly 20 includes a housing 100 composed of epoxy, molded plastic or like materials. The housing 100 further contains two connector ports 80 and 90, which generally extend from one side of the housing 100 and extend inward toward, but fall short of, the other side of the housing 100. The port 80 is comprised of first and second substantially aligned longitudinal bores 106 and 108, and likewise, the port 90 is comprised of first and second substantially longitudinal bores 110 and 112.

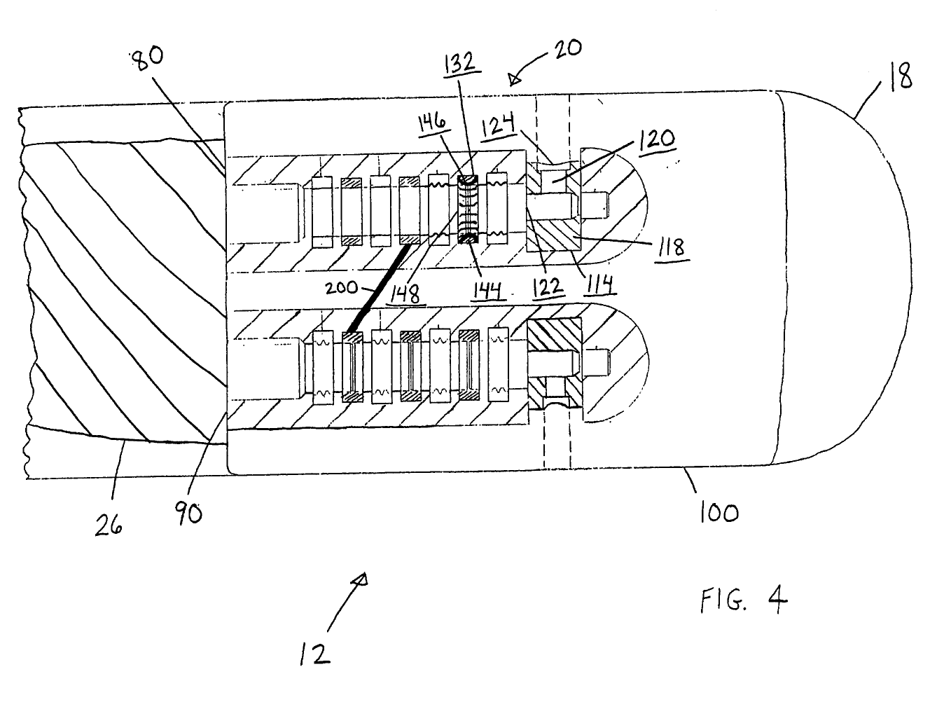

Located within each of the first longitudinal bores 106 and 110 are respective terminal blocks 114 and 116. Both the terminal blocks 114 and 116 embodied herein are similar in structure, however it is contemplated that the terminal blocks may be of differing structure, and it is not meant to limit the invention in embodying them as similar. Looking at FIG. 4 (illustrating the same embodiment as in FIG. 3), the terminal block 114 is of a type conventionally used in conjunction with implantable cardiac pacemakers and cardioverters/defibrillators, as disclosed in U.S. Pat. No. 6,006,135, issued to Kast et al., the teachings of which are incorporated herein by reference. The terminal block 114 includes a conductive connector 118 and a set screw 120. The conductive connector 118 is comprised of a pair of bores 122 and 124. Bore 122, located in the conductive connector side, is utilized for receiving a proximal end of a lead, generally referred to as the lead connector and typically comprising a terminal pin. Bore 124, located in the conductive connector top, is utilized for receiving the set screw 120. The terminal pin is then held in place by tightening the set screw 120 through the bore 124 until the set screw 120 contacts the terminal pin. Other suitable mechanisms may be used in place of the set screw 120 to secure the lead connector.

Referring back to FIG. 3, located within each of the second longitudinal bores 108 and 112 are respective spring clip contacts 132, 134, 136 and 138, 140, 142. All the spring clip contacts 132, 134, 136, 138, 140, and 142 embodied herein are similar in structure, however it is contemplated that the spring clips may be of differing structure, and it is not meant to limit the invention in embodying them as similar. Looking at FIG. 4 (illustrating the same embodiment as in FIG. 3), the spring clip contact 132 is illustrated, and includes a conductive ferrule 144 and an internal spring contact 146, of a type conventionally used in conjunction with implantable cardiac pacemakers and cardioverters/defibrillators, as disclosed in U.S. Pat. No. 5,207,218, issued to Carpentier et al., the teachings of which are incorporated herein by reference. The conductive ferrule 144, comprised of a bore 148, is used for receiving a terminal contact of a lead connector. Subsequently, the internal spring contact 146 engages the terminal contact that is stationed in the conductive ferrule 144, and in turn, provides a secure, consistent electrical contact between the internal spring contact 146, the terminal contact, and the conductive ferrule 144.

Referring back to FIG. 3, also located within each of the second longitudinal bores 108 and 112 are seals 152. All the seals 152 embodied herein are similar in structure, however it is contemplated that the seals may be of differing structure, and it is not meant to limit the invention in embodying them as similar. Seals 152 are insulated areas generally comprised of O-rings that prevent electrical conduction from occurring across their surfaces. In general, there is a seal located on both sides of each spring clip contact that electrically isolates the spring clip contact. This includes a seal located between the terminal pin and closest (i.e., first) spring clip contact in each port that electrically isolates these two connections. For example, in the first and second longitudinal bores 106 and 108 of port 80, there are four electrical contacts, the terminal block 114 and the three spring clip contacts 132, 134, and 136. Therefore, at least four seals 152 are necessary, one on each side of spring clip contacts 132, 134, and 136. Included within these four seals 152 is one that is located between terminal block 114 and the first spring clip contact 132. Similarly in the first and second longitudinal bores 110 and 112 of port 90, there are four electrical contacts being utilized, the terminal block 116 and the three spring clip contacts 138, 140, and 142. Thus, at least four seals 152 are necessary.

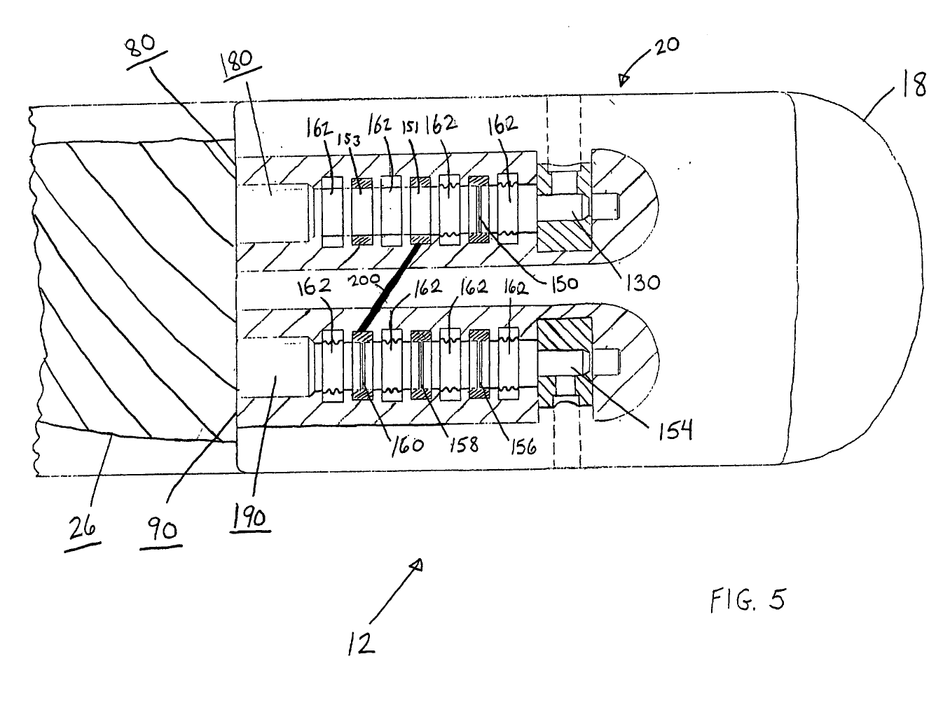

As previously mentioned with FIG. 1, the proximal ends of the leads 14 and 16 are connected to the header assembly 20 through a dual lead system plug 26. The dual lead system plug 26 is comprised of two lead connectors 180 and 190 (see FIG. 5), which respectively originate from leads 14 and 16. The lead connectors 180 and 190, in their respective ports 80 and 90, are shown in FIG. 5 (illustrating the same embodiment as in FIG. 3). As is well known in the art, each electrical contact on the surface of a lead connector is electrically connected to one distinct electrode at the distal end, generally by way of a conductor being passed within the lead body The lead connector 180 has four electrical contacts on its surface, however, only two of the electrical contacts are being utilized, a terminal pin 130 and one terminal contact 150. Herein, the terminal pin 130 is electrically connected to the tip electrode 32 (shown in FIG. 1) and the terminal contact 150 is electrically connected to the ring electrode 36 (shown in FIG. 1). The lead connector 190 has four electrical contacts on its surface and all four of the electrical contacts are being utilized, a terminal pin 154 and three terminal contacts 156, 158, and 160. Herein, the terminal pin 154 is electrically connected to the tip electrode 34 (shown in FIG. 1) and the terminal contacts 156, 158, and 160 are respectively electrically connected to ring electrode 38, coil electrode 40, and coil electrode 42 (shown in FIG. 1).

Also located on each lead connector 180 and 190 are seal zones 162, which are insulated areas that prevent electrical conduction from occurring across their surfaces. In general, there is a seal zone 162 located on both sides of each terminal contact (whether or not the contact is being utilized) that electrically isolates the terminal contacts from each other. This includes a seal zone 162 located between the terminal contact and the terminal in each port that electrically isolates these two connections. For example, on lead connector 180, there are four electrical contacts, the terminal pin 130 and the three terminal contacts 150, 151, and 153. Therefore, at least four seal zones 162 are necessary. A seal zone 162 is located on both sides of terminal contacts 150, 151, and 153, including a seal zone 162 located between the terminal pin 130 and the first (i.e., closest) terminal contact 150. On lead connector 190, there are four electrical contacts, the terminal pin 154 and the three terminal contacts 156, 158, and 160. Thus, at least four seal zones 162 are necessary, positioned similarly to those on connector 180.

From FIGS. 3 through 5 and the discussion above, a detailed illustration of the ports 80 and 90 and the corresponding lead connectors 180 and 190 for the 4-2 dual lead system is given. In referencing FIG. 5, the lead connector 180 only has two utilized electrical contacts, terminal pin 130 and terminal contact 150. Even so, the port 80 in which the lead connector 180 resides is designed with additional seals 152 to isolate high voltage contacts if necessary. Thus, if spring clip contacts 134 and 136 (FIG. 3) are utilized, and high voltage terminal contacts from a lead connector are placed therein, the port 80 is equipped with appropriate isolation. Port 90 is equipped in a similar fashion, however in the case of the connector lead 190, such high voltage isolation is necessary because of the utilization of the terminal block 116 and all of the spring clip contacts 138, 140, and 142 by the electrical contacts on the lead connector 190. By equipping the ports 80 and 90 so that all the electrical contact areas are appropriately isolated, the ICD 12 is brought closer to being compatible with differing dual lead system configurations.

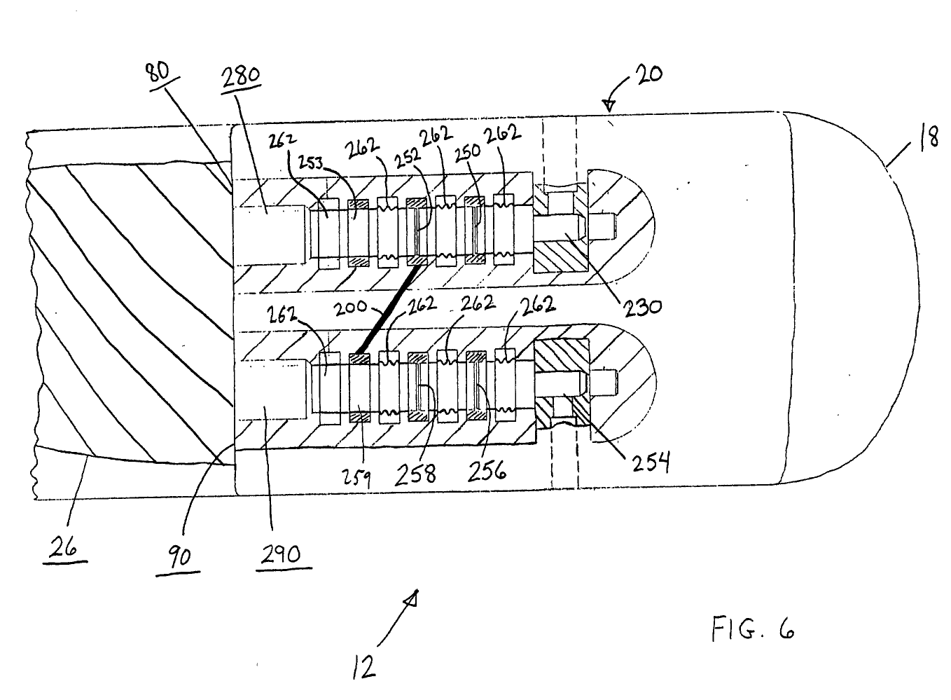

The detailed structure of the header assembly 20 and the connection thereof with proximal lead connectors of the respective leads 52 and 54 in FIG. 2 may be understood by referring now to FIG. 6, which illustrates a cross-sectional view of FIG. 2 taken along line 6-6. The header assembly 20, the housing 100, the connector ports 80 and 90, and all the elements already described above and illustrated in FIGS. 3 and 4 remain the same and will retain the same reference numbers. The only distinguishing elements between the implantable cardiac stimulator systems in FIGS. 1 and 2 are the lead systems that are being utilized.

As previously mentioned, the proximal ends of the leads 52 and 54 are connected to the header assembly 20 through a dual lead system plug 26. The dual lead system plug 26 is comprised of two lead connectors 280 and 290, which respectively originate from leads 52 and 54. The lead connectors 280 and 290, in their respective ports 80 and 90, are shown in FIG. 6. As is well known in the art, some of the electrical contacts on the surface of a lead connector are electrically connected to distinct electrode at the distal end of the lead, generally by way of conductors being passed within the lead body. The lead connector 280 has four electrical contacts on its surface, however, only three are being utilized, a terminal pin 230 and two terminal contacts 250 and 252. Herein, the terminal pin 230 is electrically connected to the tip electrode 66 (shown in FIG. 2) and the terminal contacts 250 and 252 are respectively electrically connected to the ring electrode 70 and the coil electrode 72 (shown in FIG. 2). The lead connector 290 also has four electrical contacts on its surface, however, only three are being utilized, a terminal pin 254 and two terminal contacts 256 and 258. Herein, the terminal pin 254 is electrically connected to the tip electrode 68 (shown in FIG. 2) and the terminal contacts 256 and 258 are respectively electrically connected to the ring electrode 74 and the coil electrode 76 (shown in FIG. 2).

Also located on each lead connector 280 and 290 are seal zones 262, which are insulated areas that prevent electrical conduction from occurring across their surfaces. In general, there is a seal zone 262 located on both sides of each terminal contact (whether or not it is being utilized) that electrically isolates the terminal contacts from each other. For example, on lead connector 280, there are four electrical contacts, the terminal pin 230 and the three terminal contacts 250, 252, and 253. Therefore, at least four seal zones 262 are necessary. A seal zone 262 is located on both side of terminal contacts 250, 252, and 253, including a seal zone 262 located between terminal pin 254 and the first (i.e., closest) terminal contact 250. On lead connector 290, there are also four electrical contacts, the terminal pin 254 and the three terminal contacts 256, 258, and 259. Thus, at least four seal zones 262 are necessary again, positioned similarly to those on connector 280.

From FIGS. 3, 4 and 6 and the discussion above, a detailed illustration of the ports 80 and 90 and the corresponding connector leads 280 and 290 for the 3-3 dual lead system is given. In referencing FIG. 6, the connector lead 280 has only utilized three electrical contacts, one terminal pin 230 and two terminal contacts 250 and 252. Even so, the port 80 in which the lead connector 280 resides is designed with an additional seal zone 152 to isolate additional a high voltage contact if necessary. Thus, if the spring clip contact 136 (FIG. 3) is utilized by placing a high voltage terminal contact from a lead connector therein, the port 80 is equipped with appropriate isolation. Port 90 is equipped in a similar fashion, but similar to the case of housing the connector lead 180, the lead connector 190 has only three electrical contacts being utilized, one terminal pin 254 and two terminal contacts 256 and 258. Thus, even though the spring clip contact 142 (FIG. 3) is not utilized, the port 90 is designed for high voltage isolation if necessary. Thus, by equipping the ports 80 and 90 so that all the potential electrical contact areas are appropriately isolated, the ICD 12 has been shown to be compatible with differing dual lead system configurations (e.g., 4-2 and 3-3), at least in terms of having adequate high voltage isolation to facilitate both lead system configurations.

Referencing FIGS. 3 through 6, there is also located a jumper connection 200 existing between the spring clip contacts 134 and 142. The jumper connection 200 is preferably a wire or electrical conductor manufactured from titanium or other conductive material sufficient to meet medical standards and all requirements of current transmission. In the embodiment shown, the jumper connection 200 is positioned in a channel 210 that interconnects the second bores 108 and 112 of the respective ports 80 and 90. The jumper connection 200, having been sized corresponding to the length of the channel, electrically connects spring clip contacts 134 and 142. By providing such an electrical connection between the two spring clip contacts 134 and 142, the jumper connection 200 enables the ICD 12 to facilitate high voltage at both contacts. In turn, this allows the header ports 80 and 90 to be compatible with both the dual lead system configurations, i.e., 4-2 and 3-3, illustrated in FIGS. 5 and 6. While the jumper connection 200 is shown located outside the can 18, it is still contemplated that the jumper connection 200 may be located inside the can 18, and it is not meant to limit the invention in embodying the jumper 200 as such in FIGS. 3 through 6. For instance, the electrical coupling of the spring clip contacts 134 and 142 may be performed within circuitry or other conductors internal to the can 18.

Looking at the embodiments illustrated in FIGS. 5 and 6, one can see that the respective lead connectors 180, 190 and 280, 290 shown for the respective 4-2 and 3-3 dual lead system configurations are virtually the same. The major difference between the lead connectors lies in the location of the high voltage terminal contact that provides defibrillation to the superior vena cava. While this high voltage terminal contact 160 is located on the lead connector 190 of the ventricular lead 16 in the 4-2 lead system configuration (FIG. 5), the corresponding high voltage terminal contact 252 is located on the lead connector 280 of the atrial lead in the 3-3 lead system configuration (FIG. 6). Thus, to make the ICD 12 electrically compatible with both lead system configurations (i.e., 4-2 and 3-3), the corresponding spring clip contacts 134 and 142 (for the respective high voltage terminal voltage contacts 160 and 252) had to be electrically connected. In turn, the added interconnecting channel 210 and the jumper connection 200 provided compatibility for both lead configurations.

As stated above, leads are considered to be high voltage (HV) if they contain any high voltage electrodes, otherwise, they are considered to be low voltage (LV). HV and LV electrodes are each designed for a distinct function. In the 4-2 leads system configuration, the jumper connection of the present invention will couple the HV signal on the fourth electrical contact (i.e., third terminal contact) of the ventricular lead connector to the third contact (i.e., second terminal contact) on the atrial lead connector. The atrial lead in this 4-2 leads system is providing low voltage atrial pacing. The second terminal contact in the atrial lead connector is a “dead” contact, having no associated conductor or electrode. One would obviously not want to place high voltage on a lead designed for low voltage. Such an event may cause an unsafe environment for the patient. Thus, there are safeguards in place in the present invention that guard against this scenario.

First, the terminal contact and the internal conductors in the atrial lead have sufficient electrical insulation to prevent the HV signal on the third terminal contact from interfering with the two active conductors. Thus, the two-conductor atrial LV lead is rated as HV compatible. HV compatible leads, including LV leads such as this insulated atrial lead, may be inserted into the quadrapolar connector ports that include the jumper connection of the present invention. As an additional safeguard, LV leads that are not HV compatible will be physically locked out of the quadrapolar connector port of the present invention, similar to how IS-1 connectors (LV) are locked out of DF-1 (HV) connector ports.

The embodiment illustrated herein was provided as an exemplary embodiment, and was not done so to limit the scope of the invention. While the interconnecting channel 210 and corresponding jumper connection 200 have been illustrated in FIGS. 3 through 6 as interconnecting the second bores 108, 112 and electrically coupling the spring clip contacts 134, 142 respectively, the interconnecting channel 210 and the jumper connection 200 could just as easily interconnect two other spring clip contacts to ensure compatibility for other configurations of quadrapolar dual lead systems. It will be appreciated that the present invention can take many forms and embodiments. The true essence and spirit of this invention are defined in the appended claims, and it is not intended that the embodiment of the invention presented herein should limit the scope thereof.

What is claimed is:

1. A header assembly for electrically connecting differently configured quadrapolar cardiac lead systems to a cardiac stimulator, comprising:
a housing having first and second ports extending from a side surface and adapted for sealably receiving a lead system plug, each port being comprised of first and second substantially aligned longitudinal bores, the lead system plug being comprised of at least two lead connectors, each lead connector having a terminal pin and at least three terminal contacts;
an electrically conductive terminal block positioned within each first bore, each terminal block being electrically connectable to the terminal pin of the respective lead connector, each terminal block being electrically connected to the cardiac stimulator;
at least three electrically conductive spring clip contacts positioned within each second bore, each spring clip contact being electrically connectable to a respective terminal contact, each spring clip contact being electrically connected to the cardiac stimulator;
seals located on both sides of each spring clip contact, one such seal being located between the terminal block and the spring clip contact closest to the terminal block in each port, the seals being comprised of an electrically insulating material; and
a jumper connection electrically connecting one of the spring clip contacts located in the first port to one of the spring clip contacts located in the second port.

2. The header assembly of claim 1, wherein the jumper connection is located outside a can portion of the cardiac stimulator.

3. The header assembly of claim 1, wherein the jumper connection is located within the housing of the header assembly.

4. The header assembly of claim 3, wherein the jumper connection electrically connects the spring clip contact located furthest away from the terminal block in the first port to the spring clip contact second furthest away from the terminal block in the second port.

5. The header assembly of claim 3, wherein the jumper connection is positioned within a channel formed in the housing that interconnects the second bores of each first and second port.

6. The header assembly of claim 1, wherein, in each port, a first spring clip contact is located adjacent to the terminal block, a second spring clip contact is located between the first spring clip contact and a third spring clip contact, the jumper connection electrically connecting the second spring clip contact in the first port to the third spring clip contact in the second port.

7. The header assembly of claim 1, wherein the housing is located outside a can portion of the cardiac stimulator.

8. The header assembly of claim 1, wherein one lead connector is formed on a bipolar right atrial lead and another lead connector is formed on a quadrapolar right ventricular lead.

9. The header assembly of claim 1, wherein the lead system connectors connect to a tri-polar right atrial lead and a tri-polar right ventricular lead.

10. The header assembly of claim 1, wherein each terminal pin is removably secured to the corresponding terminal block by a set screw in the terminal block.

11. The header assembly of claim 1, wherein the first and second ports are adapted to removably secure the lead system plug.

12. A header assembly for electrically connecting cardiac leads to a cardiac stimulator, comprising:
a housing having elongate ports extending inward from a side surface of the housing, each port adapted for removably receiving and sealing a lead connector, each lead connector having a terminal pin and at least three terminal contacts;
an electrically conductive terminal block positioned within each port, the terminal block electrically connecting the terminal pin of the lead thereto, the terminal block being electrically connected to the cardiac stimulator;
at least three electrically conductive spring clip contacts positioned within each port adjacent to the respective terminal block, each spring clip contact electrically connecting a respective terminal contact of the lead connector thereto, each spring clip contact being electrically connected to the cardiac stimulator;
a seal located on both sides of each spring clip contact, one such seal being located between the terminal block and the spring clip contact closest to the terminal block in each port, the seals being comprised of an electrically insulating material; and
a jumper connection in the housing electrically connecting one of the spring clip contacts located in one of the ports to one of the spring clip contacts located in another one of the ports.

13. The header assembly of claim 12, wherein the jumper connection electrically connects the spring clip contact in one of the ports located furthest away from the respective terminal block to the spring clip contact in another one of the ports second furthest away from the respective terminal block.

14. The header assembly of claim 12, wherein the jumper connection is positioned within a channel formed in the housing that interconnects two of the ports.

15. The header assembly of claim 12 wherein, in each port, a first spring clip contact is located adjacent to the terminal block, a second spring clip contact is located between the first spring clip contact and a third spring clip contact, the jumper connection electrically connecting the second spring clip contact in the one port to the third spring clip contact in the other port.

16. The header assembly of claim 12, wherein each terminal pin is removably secured to the corresponding terminal block by a set screw in the terminal block.

17. The header assembly of claim 12, wherein each seal is adapted to engage with a respective seal zone, the seal zones located on both sides of each terminal contact.

18. A method of constructing a header assembly for electrically connecting differently configured quadrapolar cardiac lead systems to a cardiac stimulator, comprising:
providing a header assembly normally configured to accept an quadrapolar lead system plug, the header assembly comprising a housing having at least two ports extending inward from a side surface, the ports being comprised of first and second substantially aligned longitudinal bores and being adapted for sealably receiving the lead system plug, the lead system plug being comprised of at least two lead connectors, each lead connector having a terminal pin and at least three terminal contacts;
opening the housing of the header assembly so that the second bores are visibly exposed, the second bores having at least three electrically conductive spring clip contacts positioned within each second bore, each spring clip contact connectable to a respective terminal contact;
providing a jumper connection;
electrically connecting one end of the jumper connection to one of the spring clip contacts located in one port and electrically connecting another end of the jumper connection to one of the spring clip contacts located in the other port; and
closing the housing of the header assembly.

19. The method of claim 18, further comprising positioning the jumper connection inside the housing.

20. The method of claim 18, further comprising positioning the jumper connection outside a can of the cardiac stimulator.

21. The method of claim 18, further comprising electrically connecting one end of the jumper connection to the spring clip contact located furthest away from the terminal block in one port and electrically connecting the other end of the jumper connection to the spring clip contact located second furthest away from the terminal block in the other port.

22. A header assembly for electrically connecting cardiac leads to a cardiac stimulator, comprising:
a housing having elongate ports extending inward from a side surface, each port adapted for removably receiving and sealing a lead connector of a cardiac lead, each lead connector having a terminal pin and at least three terminal contacts;
an electrically conductive terminal block positioned within each port, the terminal block being electrically connectable to the terminal pin of the lead, the terminal block being electrically connected to the cardiac stimulator;
at least three electrically conductive spring clip contacts positioned within each port adjacent to the respective terminal block, each spring clip contact adapted for electrically connecting a respective terminal contact of the lead connector thereto, each spring clip contact being electrically connected to the cardiac stimulator;
a seal located on both sides of each spring clip contact, one such seal being located between the terminal block and the spring clip contact closest to the terminal block in each port, the seals being comprised of an electrically insulating material; and
means for electrically connecting one of the spring clip contacts located in one of the ports to one of the spring clip contacts located in another one of the ports.
